(19) United States
(12) Patent Application Publication
(10) Pub. No.: US 2005/0211370 A1
(43) Pub. Date: Sep. 29, 2005

(54) CERAMIC DISCHARGE VESSEL WITH JOINED CAPILLARIES

(75) Inventors: Joseph V. Lima, Salem, MA (US); Victor E. Perez, Manchester, NH (US); Gregory Zaslavsky, Marblehead, MA (US); Jeffrey T. Neil, North Reading, MA (US)

Correspondence Address: OSRAM SYLVANIA INC, 100 ENDICOTT STREET, DANVERS, MA, 01923, US

(73) Assignee: OSRAM SYLVANIA INC., Danvers, MA (US)

(21) Appl. No.: 11/160,335

(22) Filed: Jun. 20, 2005

(51) Int. Cl. B32B 31/00; A61F 13/15; A61F 13/20

(52) U.S. Cl. 156/273.5

(57) ABSTRACT

A ceramic discharge vessel for a lamp includes a hollow body, two capillaries that extend from a same side of the body, and a strengthening web between and joining the two capillaries to each other.

### BACKGROUND OF THE INVENTION

The present invention is directed to a discharge vessel for a high-intensity discharge lamp. More particularly, this invention is related to ceramic discharge vessels for metal halide lamps.

A ceramic discharge vessel typically includes a ceramic body, for example of translucent polycrystalline aluminum oxide (PCA), and two ceramic capillaries that each extend from the body and receive an electrode whose discharge tip is inside the body. The electrode is sealed hermetically to its respective ceramic capillary with a frit material. The electrodes typically have multiple sections, e.g., a niobium feedthrough section welded to a tungsten shaft having a tungsten coil on its end that serves as the discharge tip. Niobium has a more favorable coefficient of thermal expansion for sealing with the ceramic capillary and tungsten has more favorable properties for sustaining the arc discharge. The capillaries are extensions that allow the frit seals to be placed far enough away from the arc discharge to keep the frit seals at a lower temperature and reduces the potential for corrosion of the frit material by reactions with the metal halide salts.

The capillaries may be placed on opposite sides of the body so that the two electrodes are collinear. Alternatively, the two capillaries may extend from the same side of the discharge vessel so that the two electrodes are side-by-side, such as disclosed in European Patent Application EP 1 111 654. The latter arrangement offers the advantage of a smaller overall length for the vessel.

### SUMMARY OF THE INVENTION

An object of the present invention is to provide a more robust ceramic discharge vessel.

A further object of the present invention is to provide a novel ceramic discharge vessel that includes a ceramic body, two capillaries that extend from a same side of the body, and a web of strengthening material between and joining the two capillaries to each other.

These and other objects and advantages of the invention will be apparent to those of skill in the art of the present invention after consideration of the following drawings and description of preferred embodiments.

### BRIEF DESCRIPTION OF THE DRAWINGS

FIG. 1 is a cross section of an embodiment of the present invention.

FIGS. 2A-D show alternative end views of the capillaries and web of embodiments of the present invention.

FIG. 3 is a pictorial representation of a further embodiment of the present invention.

FIG. 4 is a cross section of yet a further embodiment of the present invention.

FIG. 5 is a pictorial representation of another embodiment of the present invention showing the plural web sections.

### DESCRIPTION OF PREFERRED EMBODIMENTS

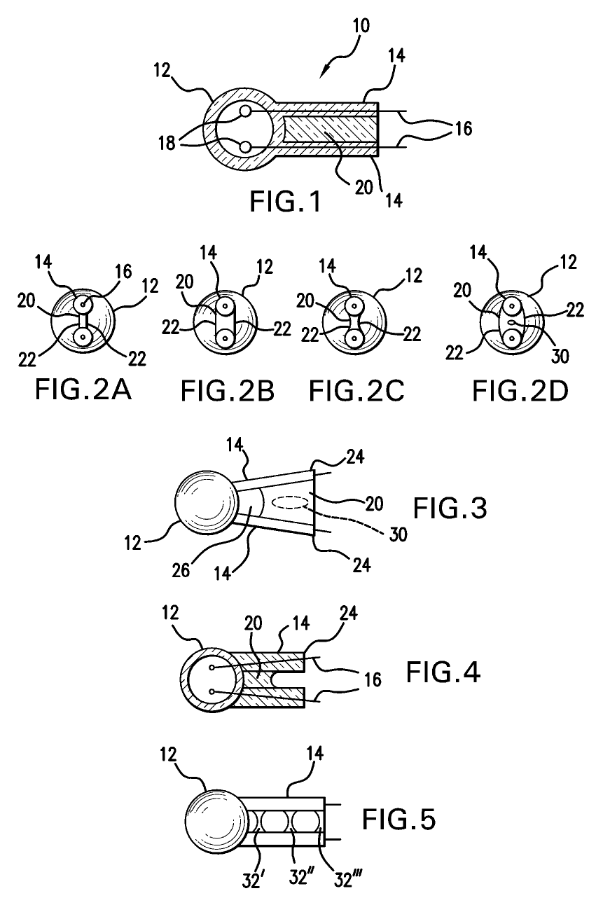

With reference now to FIG. 1, an embodiment of the present invention is a ceramic discharge vessel 10 having a hollow ceramic body 12 and two capillaries 14 that extend from a same side of body 12. In this embodiment, the body of the discharge vessel is substantially spherical so the same side refers to the same hemisphere of the ceramic body. Each of the two capillaries 14 houses a respective electrode 16 whose discharge tip 18 is inside body 12 and whose opposite end extends from the respective capillary. A web 20 of material is between and joins the two capillaries 14 to each other along most of their length. Web 20 increases the strength of discharge vessel 10 by creating, with the capillaries, a structure similar to an I-beam whose strength is superior to that of the capillaries alone.

FIGS. 2A-D show alternative end views of capillaries 14 and web 20. FIG. 2A shows that opposite sides 22 of web 20 may be parallel to each other and spaced apart a distance smaller than a diameter of capillaries 14. FIG. 2B shows that opposite sides 22 of web 20 may be parallel to each other and spaced apart a distance about the same as a diameter of capillaries 14 so that the opposite sides 22 are tangential to capillaries 14. FIG. 2C shows that the opposing surfaces 22 may converge towards each other between the two capillaries 14 so that the two capillaries and web 20 form a figure-8 in cross section. FIG. 2D shows that the opposing surfaces 22 may diverge from each other between the two capillaries 14 so that the two capillaries and web 20 form an oval in cross section.

Capillaries 14 are generally depicted herein as tubular with annular cross sections. Other cross-sectional shapes are possible, including elliptical and polygonal. Further, the web may be integral with the two capillaries, or may be a separate member. Preferably the two capillaries 14 are made of the same material as body 12, and preferably web 20 is made of the same material as the two capillaries, although other materials may be used that provide similar results. Body 20 may have any suitable shape, including spherical, elliptical, and cylindrical. Preferably, the capillaries, web and at least a portion of the body of the discharge vessel (e.g., one hemisphere) are integrally formed from a ceramic material, preferably polycrystalline aluminum oxide.

With reference to FIG. 3, the two capillaries 14 may have longitudinal axes (and respective electrodes 16) that diverge from each other (e.g., from 2-15°). This arrangement ensures that the two electrodes are closest to each other at electrode tips 18 so that the discharge will remain at the tips and not creep down the electrodes toward the body. A stable position for the arc discharge is important for focused beam applications and in all cases to prevent damage to the wall of the discharge vessel. FIG. 4 shows that the two capillaries 14 may have longitudinal axes that are parallel and yet the two electrodes 16 may have longitudinal axes that diverge from each other.

The web 20 may extend longitudinally to a length that provides the requisite strength. The preferred length of web 20 is at least half of the length of the capillaries 14. Web 20 may join the distal ends 24 of the capillaries leaving a space 26 adjacent to body 12 as shown in FIG. 3 or may leave the distal ends 24 unattached to each other as shown in FIG. 4. Web 20 may also have a longitudinal hollow space therein, such as shown in FIGS. 2D and 3 as opening 30 that extends longitudinally and that may be enclosed or open at one or both ends.

An alternative embodiment shown in FIG. 5 includes plural (preferably at least two) spaced-apart web sections 32′, 32″, 32″′ that join the two capillaries 14 to each other. The capillaries and web sections may have cross sections similar to those discussed above. Preferably, the web sections are spaced from the body of the discharge vessel and include one of the web sections at or near the distal ends of the capillaries. The plural web sections and capillaries of this embodiment form a truss-like structure that is stronger than the capillaries alone.

The discharge vessel of the present invention may be made by isopressing, injection molding, gel casting, slip casting or other known ceramic forming methods. The vessel may be made in two or more differently shaped parts which are then joined by conventional means. A first part may include half the body (e.g., a hemisphere) and the second part may include the other half of the body and the two capillaries and web. The first part may be difficult to hold and thus a small external post (not shown) may be temporarily attached to the first part to facilitate holding the first part while attaching the second part thereto. A method of joining two ceramic parts in their green state is described in U.S. Pat. No. 6,620,272, which is incorporated by reference.

While embodiments of the present invention have been described in the foregoing specification and drawings, it is to be understood that the present invention is defined by the following claims when read in light of the specification and drawings.

1. A discharge vessel for a lamp, comprising:
a hollow, ceramic body;
two ceramic capillaries that extend from a same side of said body, each of said two capillaries being arranged and adapted to receive a respective electrode that extends into said body; and
a web between and joining said two capillaries to each other along a length of said two capillaries.

2. The discharge vessel of claim 1, wherein said web is integral with and made of a same material as said two capillaries and body.

3. The discharge vessel of claim 1, wherein said web has opposing surfaces that converge towards each other between said two capillaries so that said two capillaries and said web form a figure-8 in cross section.

4. The discharge vessel of claim 1, wherein said web has opposing surfaces that are parallel to each other.

5. The discharge vessel of claim 4, wherein said opposing surfaces are tangential to said two capillaries.

6. The discharge vessel of claim 4, wherein said opposing surfaces are spaced apart by a distance less than a diameter of said capillaries.

7. The discharge vessel of claim 1, wherein said web has opposing surfaces that diverge from each other between said two capillaries so that said two capillaries and said web form an oval in cross section.

8. The discharge vessel of claim 1, wherein said two capillaries have longitudinal axes that are parallel.

9. The discharge vessel of claim 8, further comprising two electrodes that each extend through a respective one of said two capillaries, where said two electrodes have longitudinal axes that are parallel to each other.

10. The discharge vessel of claim 8, further comprising two electrodes that each extend through a respective one of said two capillaries, where said two electrodes have longitudinal axes that diverge from each other.

11. The discharge vessel of claim 1, wherein said two capillaries have longitudinal axes that diverge from each other.

12. The discharge vessel of claim 1, wherein said web is between and joins distal ends of said two capillaries to each other.

13. The discharge vessel of claim 1, wherein said web is between and joins said two capillaries to each other along their entire lengths.

14. The discharge vessel of claim 1, wherein said web has a longitudinal opening therein.

15. The discharge vessel of claim 1 wherein the web extends at least one-half the length of the capillaries.

16. The discharge vessel of claim 1 wherein the web has at least one opening.

17. The discharge vessel of claim 1 wherein the web has an opening adjacent the body.

18. A discharge vessel for a lamp, comprising:
a hollow, ceramic body;
two ceramic capillaries that extend from a same side of said body, each of said two capillaries being arranged and adapted to receive a respective electrode that extends into said body; and
plural spaced-apart web sections that are spaced from said body and between said two capillaries and that each joins said two capillaries to each other.
